(19) United States
(12) Patent Application Publication
(10) Pub. No.: US 2021/0364531 A1
(43) Pub. Date: Nov. 25, 2021

(54) Methods and Systems for Quantitative Detection of Antibodies

(71) Applicant: Laboratory Corporation of America Holdings, Burlington, NC (US)

(72) Inventor: Kelly Y. Chun, Burlington, NC (US)

(21) Appl. No.: 17/324,856

(22) Filed: May 19, 2021

Related U.S. Application Data
(60) Provisional application No. 63/027,032, filed on May 19, 2020.

(51) Int. Cl. G01N 33/68; G01N 33/569; G01N 33/543

(52) U.S. Cl. CPC G01N 33/6854; G01N 33/56983; G01N 2333/165; G01N 2469/20; G01N 33/54306

(57) ABSTRACT

The present disclosure relates to methods, systems, kits, and computer-program products for the quantification of antibodies. In an embodiment of the disclosure, the method comprises generating a standard curve and subsequently utilizing the standard curve to quantitate an antibody of interest. Also disclosed are systems, kits, and computer-program products for the quantitative detection of an antibody of interest in a sample using the methods described herein. In certain embodiments, the standard curve is developed using an immunoglobulin (e.g., IgG) that does not specifically recognize the antigen(s) recognized by the antibody of interest but is of the same immunoglobulin class as the antibody of interest (IgG). In this way, an applicable standard curve may be generated for various isolates of the antibody of interest.

### RELATED APPLICATIONS

This application claims priority to U.S. Provisional Application No. 63/027,032, filed on May 19, 2020. The entire content of said provisional application is herein incorporated by reference for all purposes.

### FIELD OF INVENTION

The present disclosure relates to the quantitative detection of antibodies.

### BACKGROUND

There is a need in the art for rapid development of assays that accurately quantitate a subject's antibody immune response to a pathogen, allergen, vaccines, drugs, or even self in autoimmunity. This assay should be precise, sensitive, and ideally be able to be run as a high-throughput screen. Such an assay would allow for studies to establish protective immunity levels to a pathogen of interest and would contribute to predicting individual prognoses and monitoring recovery following an infection with the pathogen. Additionally, the assay could be used to study humoral responses to vaccine and drug treatments, allergic reactions, autoimmunity, and to assist in the selection of donor plasma for convalescent plasma therapy. The present disclosure allows for rapid development of quantitative antibody assays in the absence of antigen-specific antibodies as a standard reagent.

### SUMMARY

The present disclosure relates to methods, systems, kits, and computer-program products for the quantitative detection of antigen specific antibodies. The methods, systems, kits, and computer-program products may be embodied in a variety of ways.

In one embodiment, the method for quantitative detection of an antibody of interest may comprise: (a) obtaining a surface coated with a binding agent, wherein the binding agent optionally is streptavidin; (b) adding a first antibody, wherein the first antibody is conjugated with a ligand that binds to the binding agent, wherein the ligand is optionally biotin, and wherein a first antibody-ligand-binding agent complex forms; (c) adding a secondary antibody such that a secondary antibody-first antibody-ligand-binding agent complex forms; (d) measuring the bound secondary antibody; (e) repeating steps (a)-(d) at various concentrations of first antibody to generate a standard curve; and (f) using the standard curve of step e) to quantify the presence of the antigen-specific antibody of interest in a sample wherein the quantification of the antibody of interest in the sample comprises the steps of: (i) adding an antigen for the antibody of interest to the surface coated with the binding agent, wherein the antigen is conjugated to the ligand, and wherein an antigen-ligand-binding agent complex forms; (ii) adding a sample comprising the antibody of interest to the surface such that an antibody of interest-antigen-ligand-binding agent complex forms; (iii) adding the secondary antibody to the surface such that a secondary antibody-antibody of interest-antigen-ligand-binding agent complex forms; (iv) measuring bound secondary antibody; and (v) quantifying the amount of bound antibody of interest using the standard curve of step e). In certain embodiments, the standard curve is developed using an immunoglobulin (e.g., IgG) that does not specifically recognize the antigen(s) recognized by the antibody of interest but is of the same immunoglobulin class as the antibody of interest (IgG). In this way, an applicable standard curve may be generated for various isolates of the antibody of interest. This can be useful as different patients may generate different specific antibodies for the antigen, but the standard curve may be used to quantify various patient sera. This can also be useful where antibodies of interest suitable for generating a standard curve are not readily available, as for example for newly identified pathogens (e.g., SARS-CoV-2).

In another embodiment, the method for quantitative detection of an antibody of interest in a sample may comprise: (a) adding an antigen recognized by the antibody of interest to a surface comprising a binding agent, wherein the binding agent binds a ligand conjugated to the antigen; (b) adding the sample to the surface such that the antibody of interest binds to the antigen to form an antibody of interest-antigen-ligand-binding agent complex; (c) adding a secondary antibody to the surface that binds the antibody of interest to form a secondary antibody-antibody of interest-antigen-ligand-binding agent complex; (d) detecting the complexed secondary antibody; and (e) quantifying the amount of bound antibody of interest based on the detected secondary antibody, wherein quantifying comprises using a standard curve generated by detecting complexes of secondary antibody-first antibody-ligand-binding agent at varying concentrations of first antibody-ligand-binding agent complexes formed under similar assay conditions, such that the first antibody substitutes for the antibody of interest when generating the standard curve. In certain embodiments, the standard curve is developed using an immunoglobulin (e.g., IgG) that does not specifically recognize the antigen(s) recognized by the antibody of interest but is of the same immunoglobulin class as the antibody of interest (IgG). In this way, an applicable standard curve may be generated for various isolates of the antibody of interest. This can be useful as different patients may generate different specific antibodies for the antigen, but the standard curve may be used to quantify various patient sera. This can also be useful where antibodies of interest suitable for generating a standard curve are not readily available, as for example for newly identified pathogens (e.g., SARS-CoV-2).

In another embodiment, the method for quantitative detection of an antibody of interest in a sample may comprise: (a) obtaining a surface coated with a binding agent, wherein the binding agent is optionally streptavidin; (b) adding to the surface an antigen that is recognized by the antibody of interest, wherein the antigen is conjugated to a ligand that binds to the binding agent, wherein the ligand is optionally biotin; (c) adding the sample to the surface such that the antibody of interest present in the sample will bind to the antigen; (d) detecting the bound antibody of interest; and e) quantifying the amount of bound antibody of interest. In certain embodiments, the standard curve used for quantifying the antibody of interest is developed using an immunoglobulin (e.g., IgG) that does not specifically recognize the antigen(s) recognized by the antibody of interest (e.g., Anti-RBD IgG) but is of the same immunoglobulin class as the antibody of interest (IgG). In this way, an applicable standard curve may be generated for various isolates of the antibody of interest. This can be useful as different patients may generate different specific antibodies for the antigen, but the standard curve may be used to quantify various patient sera. This can also be useful where antibodies of interest suitable for generating a standard curve are not readily available, as for example for newly identified pathogens.

Other embodiments of the disclosure include systems, kits, and computer-program products for quantitative detection of an antibody of interest by any of the methods disclosed herein.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a flow chart illustrating an embodiment of the disclosed methods.

FIG. 2 depicts an example of expected IgG and IgM antibody response curves following SARS-CoV-2 infection.

FIG. 3 shows an illustrative embodiment of a system in which certain embodiments of the technology may be implemented.

FIG. 4 illustrates a complexed secondary antibody-first antibody-ligand-binding agent used to generate a standard curve in accordance with an embodiment of the disclosure. In this particular embodiment, the binding agent is streptavidin, the ligand is biotin, the first antibody is affinity purified polyclonal human IgG, and the secondary antibody is Anti-IgG conjugated to HRP.

FIG. 5 illustrates a standard curve generated in accordance with an embodiment of the disclosure.

FIG. 6 illustrates a complexed secondary antibody-antibody of interest-antigen-ligand-binding agent used to quantify the antibody of interest in accordance with an embodiment of the invention. In this particular embodiment, the binding agent is streptavidin, the ligand is biotin, the antigen is RBD, the antibody of interest is patient Anti-RBD IgG, and the secondary antibody is Anti-IgG conjugated to HRP.

### DETAILED DESCRIPTION

The following description recites various aspects and embodiments of the present compositions and methods. No particular embodiment is intended to define the scope of the compositions and methods. Rather, the embodiments merely provide non-limiting examples of various methods, systems, kits, and computer-program products that are at least included within the scope of the compositions and methods. The description is to be read from the perspective of one of ordinary skill in the art; therefore, information well known to the skilled artisan is not necessarily included.

### Definitions

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Methods

The present disclosure provides a method for the quantitative detection of an antibody of interest. In an embodiment, the antibody of interest is rare such that sufficient quantities are not commercially available for use as a standard. In an embodiment of the disclosure, the method comprises generating a standard curve and subsequently utilizing the standard curve to quantitate an antibody of interest. In certain embodiments, the standard curve is developed using an immunoglobulin (e.g., IgG) that does not specifically recognize the antigen(s) recognized by the antibody of interest (e.g., an IgG specific to a particular antigen) but is of the same immunoglobulin class as the antibody of interest (IgG). In this way an applicable standard curve may be generated for various isolates of the antibody of interest. This can be useful as different subjects (e.g., patients) may generate different specific antibodies for the antigen, but the standard curve may be used to quantify antibody titers in various patient sera. In an embodiment, the quantitation of the antibody of interest is a mass (e.g., μg) of antibody for specimen volume (e.g., mL). For example, the assay may be used for detection of immune responses to new viruses or other pathogens against which large quantities of antibodies are not available. In an embodiment, the methods may be used for assaying antibodies potentially protective against SARS-CoV-2 by measurement of anti-RBD IgG present in a sample from the subject.

Also disclosed is a method for developing an assay for the quantitative detection of an antibody of interest. In an embodiment, the antibody of interest is rare such that sufficient quantities are not commercially available for use as a standard. In an embodiment of the disclosure, the method comprises generating a standard curve and subsequently utilizing the standard curve to quantitate an antibody of interest. In certain embodiments, the standard curve is developed using an immunoglobulin (e.g., IgG) that does not specifically recognize the antigen(s) recognized by the antibody of interest (e.g., Anti-RBD IgG) but is of the same immunoglobulin class as the antibody of interest (IgG). In this way an applicable standard curve may be generated for various isolates of the antibody of interest. This can be useful as different patients may generate different specific antibodies for the antigen, but the standard curve may be used to quantify antibody titers is various patient sera. In an embodiment, the quantitation of the antibody of interest is a mass (e.g., μg) of antibody for specimen volume (e.g., mL). In an embodiment, the method provides for the development of an assay that may be used for detection of immune responses to new viruses or other pathogens for which large quantities of antibodies are not available. In an embodiment, the methods may be used for assay of antibodies potentially protective against SARS-CoV-2 by measurement of anti-RBD IgG present in a sample from the subject.

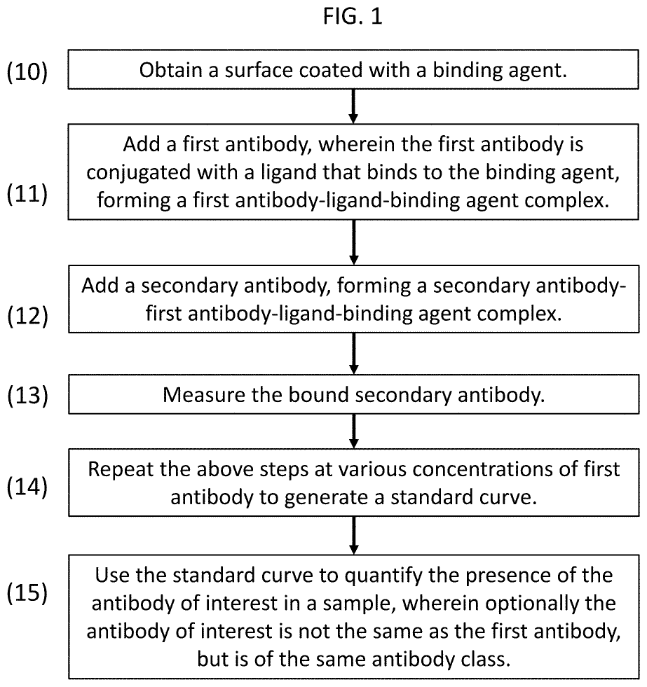

In an embodiment of the disclosure, the method comprises generating a standard curve and subsequently utilizing the standard curve to quantitate an antibody of interest. An embodiment of the method is illustrated in FIG. 1. In certain embodiments, the standard curve is developed using an immunoglobulin (e.g., IgG) that does not specifically recognize the antigen(s) recognized by the antibody of interest (e.g., Anti-RBD IgG) but is of the same immunoglobulin class as the antibody of interest (IgG). In this way an applicable standard curve may be generated for various isolates of the antibody of interest. This can be useful as different patients may generate different specific antibodies for the antigen, but the standard curve may be used to quantify various patient sera. This can also be useful where antibodies of interest suitable for generating a standard curve are not readily available, as for example for newly identified pathogens.

In order to generate the standard curve, surfaces may be first coated with a binding agent, such as streptavidin. Or, other binding agents (e.g., antibodies, antigens, receptors, and the like) may be used. Next, varying concentrations of a first antibody conjugated to a ligand for the binding agent may be added to the surfaces. Optionally, the first antibody may be purified human IgG and the ligand may be biotin. Or, antibodies specific to the classes of immunoglobulins (e.g., IgM, IgA, AgA, IgD, or IgE) may be used, depending on the antibody class of the antibody of interest. Or other ligand-binding agent combinations may be used. In an embodiment, the affinity of the ligand for the binding agent may be greater than the affinity for first antibody and the secondary antibody and/or the antibody of interest and the affinity of the secondary antibody or the antibody of interest and the antigen. For example, in certain embodiments, the affinity of the ligand for the binding agent may have a dissociation constant of up to the order of 10⁻¹⁴ mol/L, and the affinity of the first antibody and the secondary antibody and/or the antibody of interest and the secondary antibody and/or the antibody of interest and the antigen may range from approximately 10⁻⁶ mol/L to 10⁻¹² mol/L. Or, complexes of other affinities may be used.

In an embodiment, the stoichiometry of the binding between the ligand (e.g., biotin) and the binding agent (e.g., streptavidin) is known and consistent under the conditions used for the assay and generating the standard curve. Also, in an embodiment, the stoichiometry of the binding of the first antibody-ligand to the binding agent is the same as the stoichiometry of the binding of the antigen-ligand to the binding agent. Also, in an embodiment, the stoichiometry of binding of the secondary antibody to the first antibody-ligand is the same as the stoichiometry of the secondary antibody to the antibody of interest. Also, in an embodiment, the stoichiometry of the binding of the antibody of interest to the antigen-ligand is the same as the binding of the stoichiometry of the binding of the secondary antibody to the first antibody. In certain embodiments, the stoichiometry for each of these interactions is 1:1. In this way there is a 1:1 correlation between the standard curve and the assay of the sample. In alternate embodiments, the relative stoichiometries may be different and a correction for these differences is made.

Next, a secondary antibody that recognizes both the antibody of interest and the primary antibody (e.g., anti-IgG where the antibody of interest and the primary antibody are both IgG) is added. The secondary antibody may be conjugated to a detectable moiety. Optionally, the secondary antibody may be anti-human IgG and the detectable moiety may be an enzyme such as, but not limited to horseradish peroxidase (HRP), alkaline phosphatase (AP), beta-galactosidase, or luciferase. Optionally, chemiluminescent detection methods may be used to detect the detectable moiety. Optionally, colormetric detection methods may be used to detect the detectable moiety. Upon measurement of the detectable moiety, a standard curve may be generated correlating the known concentrations of the first antibody and each's respective detectable moiety measurement.

This standard curve can be used with measurements taken from a comparable assay to quantify an antibody of interest in a patient sample. For the quantitation assay, surfaces may be first coated with the binding agent used to generate the standard curve, e.g. streptavidin. Next, an antigen for the antibody of interest conjugated to the ligand for the binding agent may be added to the surfaces. For example, for assay of antibodies potentially protective against SARS-CoV-2, the antigen may be SARS-CoV-2 receptor binding domain (RBD) and the ligand may be biotin. Next, the sample containing the antibody of interest may be added to the surfaces. Optionally, the antibody of interest may be anti-SARS-CoV-2 RBD IgG. Finally, the secondary antibody, e.g. anti-human IgG, conjugated to the detectable moiety, e.g. HRP, may be added to the surfaces. Upon measurement of the detectable moiety, the standard curve can be used to calculate the concentration of the antibody of interest in the sample.

In one embodiment, the method for quantitative detection of an antibody of interest may comprise: (a) obtaining a surface coated with a binding agent, wherein the binding agent optionally is streptavidin; (b) adding a first antibody, wherein the first antibody is conjugated with a ligand that binds to the binding agent, wherein the ligand is optionally biotin, and wherein a first antibody-ligand-binding agent complex forms; (c) adding a secondary antibody such that a secondary antibody-first antibody-ligand-binding agent complex forms; (d) measuring the bound secondary antibody; (e) repeating steps (a)-(d) at various concentrations of first antibody to generate a standard curve; and (f) using the standard curve of step (e) to quantify the presence of the antibody of interest in a sample wherein the quantification of the antibody of interest in the sample comprises the steps of: (i) adding an antigen for the antibody of interest to the surface coated with the binding agent, wherein the antigen is conjugated to the ligand, and wherein an antigen-ligand-binding agent complex forms; (ii) adding a sample comprising the antibody of interest to the surface such that an antibody of interest-antigen-ligand-binding agent complex forms; (iii) adding the secondary antibody to the surface such that a secondary antibody-antibody of interest-antigen-ligand-binding agent complex forms; (iv) measuring bound secondary antibody; and (v) quantifying the amount of bound antibody of interest using the standard curve of step (e). In certain embodiments, the standard curve used for quantifying the antibody of interest is developed using an immunoglobulin that does not specifically recognize the antigen(s) recognized by the antibody of interest but is of the same immunoglobulin class as the antibody of interest.

In another embodiment, the method for quantitative detection of an antibody of interest in a sample may comprise: (a) adding an antigen recognized by the antibody of interest to a surface comprising a binding agent, wherein the binding agent binds a ligand conjugated to the antigen; (b) adding the sample to the surface such that the antibody of interest binds to the antigen to form an antibody of interest-antigen-ligand-binding agent complex; (c) adding a secondary antibody to the surface that binds the antibody of interest to form a secondary antibody-antibody of interest-antigen-ligand-binding agent complex; (d) detecting the complexed secondary antibody; and (e) quantifying the amount of bound antibody of interest based on the detected secondary antibody, wherein quantifying comprises using a standard curve generated by detecting complexes of secondary antibody-a first antibody at varying concentrations-ligand-binding agent complexes formed under similar assay conditions. In certain embodiments, the standard curve used for quantifying the antibody of interest is developed using an immunoglobulin that does not specifically recognize the antigen(s) recognized by the antibody of interest but is of the same immunoglobulin class as the antibody of interest.

In another embodiment, the method for quantitative detection of an antibody of interest in a sample may comprise: (a) obtaining a surface coated with a binding agent, wherein the binding agent is optionally streptavidin; (b) adding to the surface an antigen that is recognized by the antibody of interest, wherein the antigen is conjugated to a ligand that binds to the binding agent, wherein the ligand is optionally biotin; (c) adding the sample to the surface such that the antibody of interest present in the sample will bind to the antigen; (d) detecting the bound antibody of interest; and (e) quantifying the amount of bound antibody of interest. In this embodiment, the step of quantifying the amount of bound antibody of interest may utilize a standard curve generated as described above.

For example, in certain embodiments, the surface is coated with streptavidin and then biotinylated RBD (the antigen) is added. The surface is then washed and a portion of a sample believed to contain anti-SARS-CoV-2 RBD IgG (i.e., the antibody of interest) is added and allowed to incubate such that the anti-SARS-CoV-2 RBD IgG complexes to the RBD antigen that is bound to the surface. After washing, the anti-SARS-CoV-2 RBD IgG-RBD bound to the surface may be detected with the anti-IgG secondary antibody. The amount of anti-SARS-CoV-2 RBD IgG-RBD bound to the surface is then quantitated based on the amount of secondary antibody detected.

In some embodiments of the disclosure, the sample is from a subject recovering from an infection with a pathogen. In some embodiments of the disclosure, the method further comprises determining if a sample from a convalescent subject contains enough antibody of interest for the subject's plasma to be used as donor plasma.

In some embodiments of the disclosure, the method further comprises using the quantified amount of antibody of interest to determine a protective immunity level of the antibody of interest.

In some embodiments of the disclosure, the method further comprises quantifying the antibody of interest at a first time point and at least a second time point to generate serial titers of the antibody of interest. In some embodiments, the serial titers may be used to track a subject's recovery from an infection with a pathogen. In some embodiments, the serial titers may be used to monitor a subject's response to a treatment. In some embodiments, the serial titers may be used to monitor a subject's response to a vaccine. In some embodiments, the serial titers may be used to assist in contact tracing applications.

In some embodiments of the disclosure, the method further comprises predicting a patient's prognosis using the quantified amount of the antibody of interest.

It will be understood that while the disclosure is exemplified in certain embodiments as a quantitative assay for anti-SARS-CoV-2 RBD IgG, the principle may be applied to the assay of other antibodies of interest. Further, it will be understood that the assay is not limited to the assay of IgG antibodies, but that it may be applied to other classes of antibodies (e.g., IgM, IgE, IgD, IgA) so long as the first antibody and the antibody of interest are both recognized to a similar extent by the secondary antibody (i.e., the secondary antibody has a similar binding affinity for the first antibody and the antibody of interest).

Binding Agents and Ligands

In some embodiments of the method, a binding agent bound to a solid surface and its ligand comprise a binding pair. Binding pairs are well known in the art. In some cases, the binding pair can consist of an antigen and an antibody that can bind to the antigen. In some cases, the binding pair can consist of a receptor and a ligand. In some cases, the binding pair can consist of an enzyme and an inhibitor of the enzyme. In some cases, the binding pair can consist of an enzyme and its co-factor. Specific examples of such specific binding pairs include, but are not limited to, carbohydrate and lectin, biotin and avidin or streptavidin or NeutrAvidin, amine-modified oligos and carboxylate, folic acid and folate binding protein, vitamin B12 and intrinsic factor, protein A and immunoglobulin, and Protein G and immunoglobulin. Either member of a binding pair can be bound to the solid surface.

In some embodiments of the method, the binding agent may be streptavidin. In some embodiments, the binding agent may be selected from streptavidin, avidin, and neutrAvidin. In some embodiments of the disclosure, the binding agent is bound to the surface at a high density.

In some embodiments of the method, the ligand may be biotin.

Antibodies

In some embodiments of the method, the first antibody may be purified IgG. In some embodiments of the method, the first antibody may be purified IgA. In some embodiments of the method, the first antibody may be purified IgM. In these embodiments, the purified immunoglobulin may be a human immunoglobulin.

In some embodiments of the method, the secondary antibody may be labeled with a detectable moiety. In some embodiments, the detectable moiety may be an enzyme. In some embodiments, the detectable moiety may be horseradish peroxidase (HRP). In some embodiments, the detectable moiety may be alkaline phosphatase (AP). In some embodiments, the detectable moiety may be a fluorophore or chemiluminescence.

“Antibody” means an immunoglobulin that binds to, and is thereby defined as complementary with, a particular spatial and polar organization of another molecule. The antibody can be monoclonal, polyclonal or recombinant and can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for binding. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′ and the like. Antibodies may also be single-chain antibodies, chimeric antibodies, humanized antibodies or any other antibody derivative known to one of skill in the art that retains binding activity that is specific for a particular binding site. In addition, aggregates, polymers and conjugates of immunoglobulins or their fragments can be used where appropriate so long as binding affinity for a particular binding site is maintained. Guidance in the production and selection of antibodies and antibody derivatives for use in immunoassays, including such assays employing releasable molecular tags (as described below) can be found in readily available texts and manuals, e.g., Harlow and Lane, 1988, Antibodies: A Laboratory Manual, Cold Spring Harbor Laboratory Press, New York; Howard and Bethell, 2001, Basic Methods in Antibody Production and Characterization, CRC Press; Wild, ed., 1994, The Immunoassay Handbook, Stockton Press, New York.

Antigens and Antibodies of Interest

Antigens may be assayed using the disclosed methods and systems.

In some embodiments, the antigen may be a bacterial antigen.

In some embodiments, the antigen may be a drug, a vaccine, a hormone, an allergen, an or an endogenous protein. In some embodiments, the antigen may be a viral antigen. In some embodiments, the antigen for the antibody of interest may be a viral receptor binding domain. In some embodiments, the antigen may be the receptor binding domain (RBD) of SARS-CoV-2. SARS-CoV-2 has four structural proteins, the spike protein, the nucleocapsid protein, the envelope protein, and the membrane glycoprotein. The spike protein comprises subunits S1 and S2. SARS-CoV-2 RBD is the part of S1 that binds to the host cell ACE2 receptor for viral entry. SARS-CoV-2 RBD is highly antigenic, and antibodies against SARS-CoV-2 RBD have been shown to neutralize and inhibit viral infections in vitro.

In some embodiments, the antibody of interest may be an anti-bacterial antigen antibody. In some embodiments, the antibody of interest may be an anti-viral antigen antibody.

In some embodiments of the method, the antibody of interest may be an anti-receptor binding domain (RBD) IgG. In some embodiments, the antibody of interest may be anti-RBD IgM. In some embodiments, the antibody of interest may be anti-RBD IgA.

In some embodiments, the antibody of interest may be anti-SARS-CoV-2 RBD IgG. In some embodiments, the antibody of interest may be anti-SARS-CoV-2 RBD IgM. In some embodiments, the antibody of interest may be anti-SARS-CoV-2 RBD IgA.

In some embodiments, the assay is specific to the antibody of interest and does not detect other antibodies that may be present in individuals exposed to a particular microbe (e.g., virus, bacteria or other). For example, for SARS-CoV-2, embodiments of the method do not detect the presence of antibodies reactive to a human coronavirus selected from the group consisting of: 229E, NL63, OC43, HKU1, and MERS-CoV.

In some embodiments, the method may further comprise determining that a person has mounted an immune response to the pathogen that results in the antibody response (e.g., SARS-CoV-2). In some embodiments, the method may further comprise determining that a person has not mounted an immune response to the pathogen that results in the antibody response (e.g., SARS-CoV-2). In some embodiments, a specific threshold (i.e., a quantitative amount such as micrograms/mL) of antibody of interest may be identified to determine whether a person has mounted an immune response to SARS-CoV-2. In some embodiments, this threshold can be optionally about 2 μg/ml, about 3 μg/ml, about 4 μg/ml, about 5 μg/ml, or about 6 μg/ml.

Detectable Moieties

Below are described some non-limiting examples of some detectable moieties that may be used.

Fluorescent Dyes

In certain embodiments, a detectable moiety is a fluorescent dye. Numerous known fluorescent dyes of a wide variety of chemical structures and physical characteristics are suitable for use in the practice of the disclosure. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), hexachloro-fluorescein (HEX), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Q-DOTS, Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, CY-5, CY-3.5, CY5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92:4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described, for example, in U.S. Pat. No. 5,945,526 to Lee et al., the entire contents of which are herein incorporated by reference. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

Enzymes

In certain embodiments, a detectable moiety is an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a molecule using a linker group such as a carbodiimide, a diisocyanate, a glutaraldehyde, and the like.

Radioactive Isotopes

In certain embodiments, a detectable moiety is a radioactive isotope. For example, a molecule may be isotopically-labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (i.e., 3H, 13C, 14C, 18F, 19F, 32P, 35S, 64Cu, 67Cu, 67Ga, 90Y, 99mTc, 111In, 125I, 123I, 129I, 131I, 135I, 186Re, 187Re, 201Tl, 212Bi, 213Bi, 211At, 153Sm, 177Lu).

Dendrimers

In some embodiments, signal amplification is achieved using labeled dendrimers as the detectable moiety (see, e.g., Physiol Genomics 3:93-99, 2000), the entire contents of which are herein incorporated by reference in their entirety. Fluorescently labeled dendrimers are available from Genisphere (Montvale, N.J.). These may be chemically conjugated to the oligonucleotide primers by methods known in the art.

Samples

In some embodiments of the method, the sample may be a serum sample. In other embodiments of the method, the sample may be a dried blood spot. In other embodiments of the method, the sample may be from a nasal swab.

“Sample” or “patient sample” or “biological sample” or “specimen” are used interchangeably herein. The source of the sample may be solid tissue as from a fresh tissue, frozen and/or preserved organ or tissue or biopsy or aspirate. The source of the sample may be a liquid sample. Non-limiting examples of liquid samples, include, blood or a blood product (e.g., serum, plasma, or the like), urine, nasal swabs, biopsy sample (e.g., liquid biopsy for the detection of cancer), a liquid sample described above, the like or combinations thereof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, or other tissue sampler other liquid aspirate. In an embodiment, the sample body fluid may be separated on the substrate prior to drying. For example, blood may be deposited onto a sampling paper substrate which limits migration of red blood cells allowing for separation of the blood plasma fraction prior to drying in order to produce a dried plasma sample for analysis.

Subjects

In some embodiments, the subject may be a human subject.

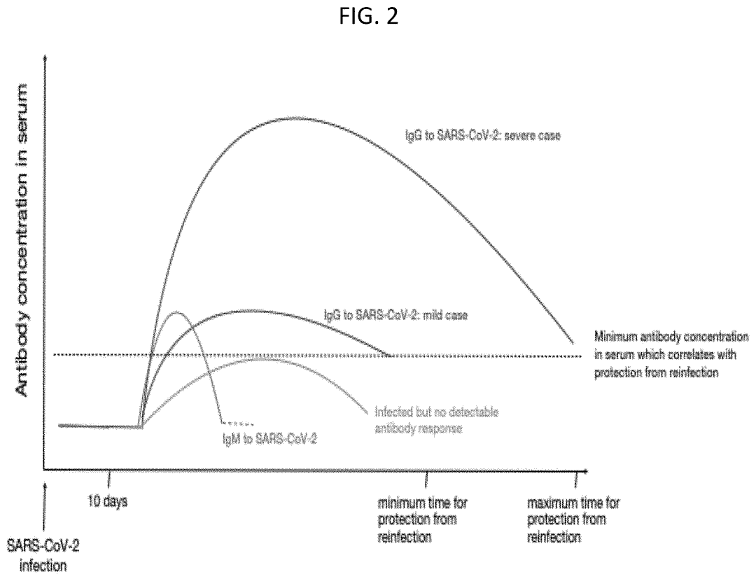

In some embodiments of the method, the subject may be suspected to have been exposed to any pathogen of interest. In certain embodiments, the pathogen is SARS-CoV-2. An example of expected antibody response curves following SARS-CoV-2 infection is depicted in FIG. 2. The average time to seroconversion following infection is 10-14 days. IgG and IgM become detectable around the same time, but the IgM response becomes undetectable after approximately two months. Conversely, the IgG response typically has not peaked at 2 months, and may persist at detectable levels for up to two years.

In some embodiments, the sample may be taken within three weeks of the subject's suspected exposure to SARS-CoV-2. In some embodiments, the sample may be taken within two weeks of the subject's suspected exposure to SARS-CoV-2. In some embodiments, the sample may be taken more than two weeks after the subject's suspected exposure to SARS-CoV-2. In some embodiments, the sample may be taken up to two months after the subject's suspected exposure to SARS-CoV-2. In some embodiments, the sample may be taken up to one year after the subject's suspected exposure to SARS-CoV-2. In some embodiments, the sample may be taken up to two years after the subject's suspected exposure to SARS-CoV-2.

As used herein, the terms “subject” and “patient” are used interchangeably. As used herein, the terms “subject” and “subjects” refer to an animal, preferably a mammal including a non-primate (e.g., a cow, pig, horse, donkey, goat, camel, cat, dog, guinea pig, rat, mouse or sheep) and a primate (e.g., a monkey, such as a cynomolgus monkey, gorilla, chimpanzee or a human). In some embodiments, a patient is a subject undergoing medical assessment or treatment.

### Systems, Kits, and Computer-Program Products

Other embodiments of the disclosure include systems and kits for quantitative detection of an antibody of interest by any of the methods disclosed herein.

In another embodiment, the disclosure comprises a system and/or a kit comprising components for quantitating an antibody of interest from a biological sample. The system and/or kit may comprise a surface coated with a binding agent. The system and/or kit may also comprise a first antibody complexed to a ligand for the binding agent. Also, the system and/or kit may comprise a secondary antibody. The system and/or kit may also comprise a station and/or instructions for generating a standard curve by methods described herein. The system and/or kit may also comprise reagents for conjugating an antibody of interest to the ligand and/or a station and/or instructions for quantitating the antibody of interest using the standard curve by methods described herein.

In one embodiment, the system or kit for quantitative detection of an antibody of interest by methods that may comprise at least one station (e.g., system) or a component (e.g., kit) for performing at least one of the following steps: (a) obtaining or generating a surface coated with a binding agent, wherein the binding agent optionally is streptavidin; (b) adding a first antibody, wherein the first antibody is conjugated with a ligand that binds to the binding agent, wherein the ligand is optionally biotin, and wherein a first antibody-ligand-binding agent complex forms; (c) adding a secondary antibody such that a secondary antibody-first antibody-ligand-binding agent complex forms; (d) measuring the bound secondary antibody; (e) repeating steps (a)-(d) at various concentrations of first antibody to generate a standard curve; and (f) using the standard curve of step e) to quantify the presence of the antibody of interest in a sample wherein the quantification of the antibody of interest in the sample comprises the steps of: (i) adding an antigen for the antibody of interest to the surface coated with the binding agent, wherein the antigen is conjugated to the ligand, and wherein an antigen-ligand-binding agent complex forms; (ii) adding a sample comprising the antibody of interest to the surface such that an antibody of interest-antigen-ligand-binding agent complex forms; (iii) adding the secondary antibody to the surface such that a secondary antibody-antibody of interest-antigen-ligand-binding agent complex forms; (iv) measuring bound secondary antibody; and (v) quantifying the amount of bound antibody of interest using the standard curve of step e). In certain embodiments, the standard curve used for quantifying the antibody of interest is developed using an immunoglobulin that does not specifically recognize the antigen(s) recognized by the antibody of interest but is of the same immunoglobulin class as the antibody of interest. In certain embodiments, at least one of the steps of the kit and or stations of the system may be controlled by a computer.

In another embodiment, the system or kit for quantitative detection of an antibody of interest in a sample may comprise at least one station (e.g., system) or a component or reagents (e.g., kit) for performing at least one of the following steps: (a) adding an antigen recognized by the antibody of interest to a surface comprising a binding agent, wherein the binding agent binds a ligand conjugated to the antigen; (b) adding the sample to the surface such that the antibody of interest binds to the antigen to form an antibody of interest-antigen-ligand-binding agent complex; (c) adding a secondary antibody to the surface that binds the antibody of interest to form a secondary antibody-antibody of interest-antigen-ligand-binding agent complex; (d) detecting the complexed secondary antibody; and (e) quantifying the amount of bound antibody of interest based on the detected secondary antibody, wherein quantifying comprises using a standard curve generated by detecting complexes of secondary antibody-first antibody-ligand-binding agent at varying concentrations of first antibody-ligand-binding agent complexes formed under similar assay conditions, such that the first antibody substitutes for the antibody of interest when generating the standard curve. In certain embodiments, the standard curve used for quantifying the antibody of interest is developed using an immunoglobulin that does not specifically recognize the antigen(s) recognized by the antibody of interest but is of the same immunoglobulin class as the antibody of interest. In certain embodiments, at least one of the steps of the kit and or stations of the system may be controlled by a computer.

In another embodiment, the system or kit for quantitative detection of an antibody of interest in a sample by methods that may comprise at least one station (e.g., system) or a component or reagents (e.g., kit) for performing at least one of the following steps: (a) obtaining a surface coated with a binding agent, wherein the binding agent is optionally streptavidin; (b) adding to the surface an antigen that is recognized by the antibody of interest, wherein the antigen is conjugated to a ligand that binds to the binding agent, wherein the ligand is optionally biotin; (c) adding the sample to the surface such that the antibody of interest present in the sample will bind to the antigen; (d) detecting the bound antibody of interest; and (e) quantifying the amount of bound antibody of interest. In certain embodiments, at least one of the steps of the kit and or stations of the system may be controlled by a computer.

In some embodiments, the system and/or kit further comprises a computer and/or a data processor. As disclosed herein, in certain embodiments, the system may comprise one or more computers, and/or a computer product tangibly embodied in a non-transitory computer readable storage medium containing instructions which, when executed on the one or more data processors, cause the one or more data processors to perform actions for performing the methods or implementing the systems and/or kits of any of embodiments disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines. For example, as disclosed below, the system and/or kits may comprise a computer and/or computer-program product tangibly embodied in a non-transitory machine-readable storage medium for plotting the standard curve and quantitating an antibody of interest. Thus, in certain embodiments, the system and/or kit may comprise components to quantify the measurement. Also, the system and/or kit may comprise components to perform statistical analysis of the data.

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein is a computer system configured to perform the any of the embodiments of the methods, or particular steps of any of the methods for quantitative detection of an antibody of interest. In some embodiments, this invention provides a system for quantitative detection of an antibody of interest comprising one or more processors and non-transitory machine readable storage medium and/or memory coupled to one or more processors, and the memory or the non-transitory machine readable storage medium encoded with a set of instructions configured to perform a process.

Also provided herein are computer readable storage media with an executable program stored thereon, where the program instructs a microprocessor to perform any of the methods or method steps described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

In some embodiments, the invention provides a non-transitory machine readable storage medium comprising program instructions that when executed by one or more processors cause the one or more processors to perform any of the methods disclosed herein.

Thus, also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Antibody quantities, concentrations, levels and/or measurements sometimes are referred to as “data” or “data sets.” In certain embodiments, data or data sets can be organized into a matrix having two or more dimensions based on one or more features or variables. Data organized into matrices can be organized using any suitable features or variables. In certain embodiments, data sets characterized by one or more features or variables sometimes are processed after counting.

Machines, software and interfaces may be used to conduct any steps of the methods and/or to generate any of the compositions described herein. Using machines, software and interfaces, a user may enter, request, query or determine options for using particular information, programs or processes, which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines and/or stations for performing certain steps of the disclosed methods or for generating the disclosed compositions. Each machine may comprise one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, cloud or web-based systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, simulated data is generated by an in silico process and the simulated data serves as data that can be input via an input device. The term “in silico” refers to research and experiments performed using a computer.

A system may include software useful for performing a process or part of a process described herein, and software can include one or more modules for performing such processes (e.g., generating a standard curve, determining a quantitative amount of the antibody of interest, and data display). The term “software” refers to computer readable program instructions that, when executed by a computer, perform computer operations. Instructions executable by the one or more microprocessors sometimes are provided as executable code, that when executed, can cause one or more microprocessors to implement a method described herein.

A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product may be embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation or genetic alteration.

A machine, in some embodiments, comprises at least one microprocessor for carrying out the instructions in a module. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like), the world wide web (www), the internet, a computer and/or another module.

Software comprising program instructions often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, FORTRAN, and the like. In some embodiments, an algorithm can be configured or modified to include margin of errors, statistical analysis, statistical significance, and/or comparison to other information or data sets (e.g., applicable when using, for example, algorithms to analyze a library of cell-free nucleic acid fragments, such as a fixed cutoff algorithm, a dynamic clustering algorithm, or an individual polymorphic nucleic acid target threshold algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity. An algorithm with the highest sensitivity and/or specificity may be identified and utilized.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

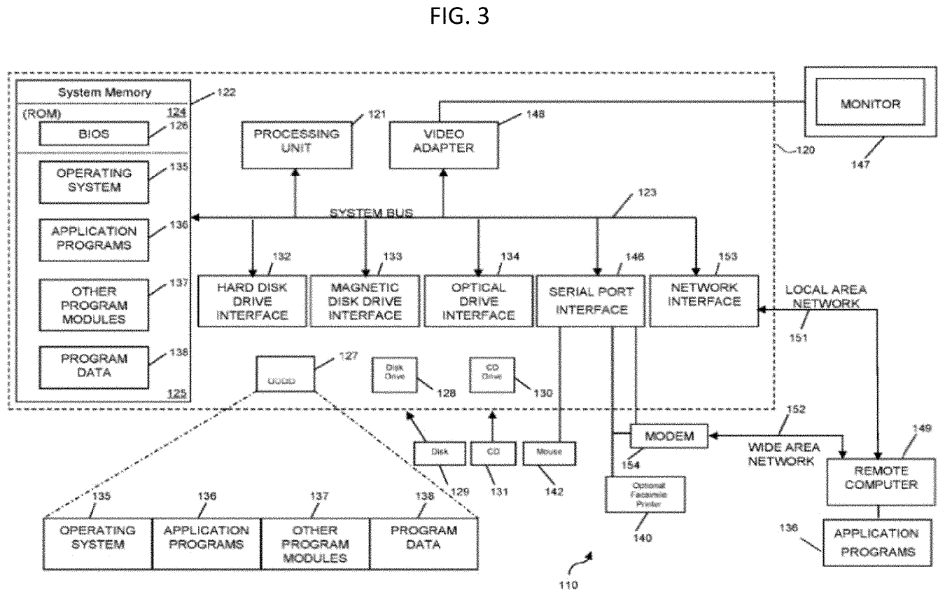

FIG. 3 illustrates a non-limiting example of a computing environment 110 in which various systems, methods, algorithms, and data structures described herein may be implemented. The computing environment 110 is only one example of a suitable computing environment and is not intended to suggest any limitation as to the scope of use or functionality of the systems, methods, and data structures described herein. Neither should computing environment 110 be interpreted as having any dependency or requirement relating to any one or combination of components illustrated in computing environment 110. A subset of systems, methods, and data structures shown in FIG. 3 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The operating environment 110 of FIG. 3 includes a general purpose computing device in the form of a computer 120, including a processing unit 121, a system memory 122, and a system bus 123 that operatively couples various system components including the system memory 122 to the processing unit 121. There may be only one or there may be more than one processing unit 121, such that the processor of computer 120 includes a single central-processing unit (CPU), or a plurality of processing units, commonly referred to as a parallel processing environment. The computer 120 may be a conventional computer, a distributed computer, or any other type of computer.

The system bus 123 may be any of several types of bus structures including a memory bus or memory controller, a peripheral bus, and a local bus using any of a variety of bus architectures. The system memory may also be referred to as simply the memory, and includes read only memory (ROM) 124 and random access memory (RAM). A basic input/output system (BIOS) 126, containing the basic routines that help to transfer information between elements within the computer 120, such as during start-up, is stored in ROM 124. The computer 120 may further include a hard disk drive interface 127 for reading from and writing to a hard disk, not shown, a magnetic disk drive 128 for reading from or writing to a removable magnetic disk 129, and an optical disk drive 130 for reading from or writing to a removable optical disk 131 such as a CD ROM or other optical media.

The hard disk drive 127, magnetic disk drive 128, and optical disk drive 130 may be connected to the system bus 123 by a hard disk drive interface 132, a magnetic disk drive interface 133, and an optical disk drive interface 134, respectively. The drives and their associated computer-readable media provide nonvolatile storage of computer-readable instructions, data structures, program modules and other data for the computer 120. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 129, optical disk 131, ROM 124, or RAM, including an operating system 135, one or more application programs 136, other program modules 137, and program data 138. A user may enter commands and information into the personal computer 120 through input devices such as a keyboard 140 and pointing device 142. Other input devices (not shown) may include a microphone, joystick, game pad, satellite dish, scanner, or the like. These and other input devices are often connected to the processing unit 121 through a serial port interface 146 that is coupled to the system bus, but may be connected by other interfaces, such as a parallel port, game port, or a universal serial bus (USB). A monitor 147 or other type of display device may be connected to the system bus 123 via an interface, such as a video adapter 148. In addition to the monitor, computers typically include other peripheral output devices (not shown), such as speakers and printers.

The computer 120 may operate in a networked environment using logical connections to one or more remote computers, such as remote computer 149. These logical connections may be achieved by a communication device coupled to or a part of the computer 120, or in other manners. The remote computer 149 may be another computer, a server, a router, a network PC, a client, a peer device or other common network node, and typically includes many or all of the elements described above relative to the computer 120, although only a memory storage device 150 has been illustrated in FIG. 3. The logical connections depicted in FIG. 3 include a local-area network (LAN) 151 and a wide-area network (WAN) 152. Such networking environments are commonplace in office networks, enterprise-wide computer networks, intranets and the Internet, which all are types of networks.

When used in a LAN-networking environment, the computer 120 is connected to the local network 151 through a network interface or adapter 153, which is one type of communications device. When used in a WAN-networking environment, the computer 120 often includes a modem 154, a type of communications device, or any other type of communications device for establishing communications over the wide area network 152. The modem 154, which may be internal or external, is connected to the system bus 123 via the serial port interface 146. In a networked environment, program modules depicted relative to the personal computer 120, or portions thereof, may be stored in the remote memory storage device. It is appreciated that the network connections shown are non-limiting examples and other communications devices for establishing a communications link between computers may be used.

The following examples of specific embodiments of the invention are offered for illustrative purposes only, and are not intended to limit the scope of the invention in any way.

### EXAMPLES

### Example 1

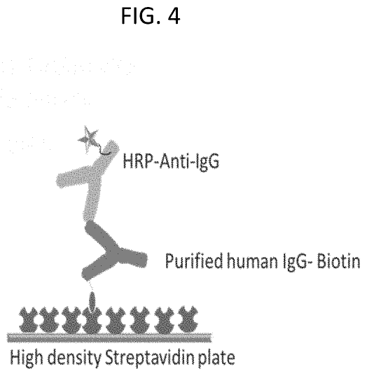

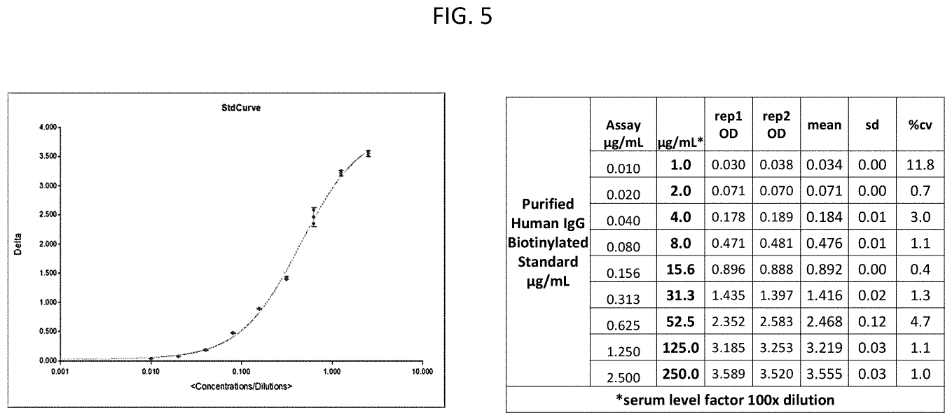

A standard curve was created using the assay setup depicted in FIG. 4. Briefly, streptavidin coated plates were incubated with concentrations of purified human IgG conjugated to biotin. The concentration of purified human IgG used to generate the standard curve ranged from 0.010 μg/ml to 2.500 μg/ml-0.010 μg/ml, 0.020 μg/ml, 0.040 μg/ml, 0.080 μg/ml, 0.156 μg/ml, 0.313 μg/ml, 0.625 μg/ml, 1.250 μg/ml, and 2.500 μg/ml. As serum was diluted 100× for this assay, these standards correlated with serum levels of IgG ranging from 1.0 ug/ml to 250.0 ug/ml. Plates were then incubated with anti-human IgG conjugated to HRP. Upon detection of HRP, a standard curve was created using the data collected at each concentration of the standard, as is shown in FIG. 5.

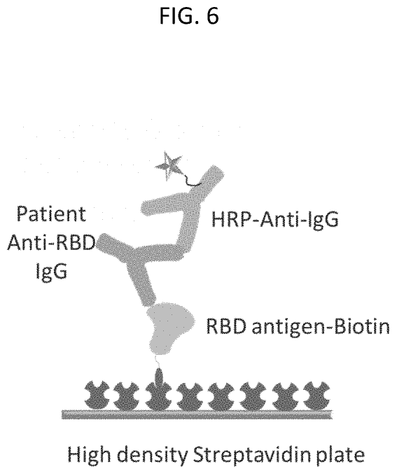

Serum samples were analyzed using the assay setup depicted in FIG. 6. Briefly, streptavidin coated plates were incubated with SARS-CoV-2 receptor binding domain (RBD) conjugated to biotin. Plates were then incubated with serum samples in duplicate from patients suspected of having been exposed to SARS-CoV-2; these patient samples were SARS-CoV-2 RNA and IL-6 positive. Plates were also incubated with control known positive and known negative serum samples in duplicate. Then plates were incubated with anti-human IgG conjugated to HRP. Upon detection, the quantity of anti-RBD IgG was determined for each sample using the standard curve as shown in Table 1.

TABLE 1

| | rep1 | rep2 | avg | |
|---|---|---|---|---|
| sample | (μg/ml) | (μg/ml) | (μg/ml) | % CV |
| Positive 2 | 17.9 | 19.6 | 18.8 | 6.4 |
| Positive 6 | 26.4 | 32.7 | 29.6 | 15.1 |
| Positive 6 | 38.9 | 34.7 | 36.8 | 8.1 |
| Positive 4 | 51.9 | 45.6 | 48.8 | 9.1 |
| Negative 10 | 1.7 | 1.3 | 1.5 | 18.9 |
| Negative 9 | 2.5 | 2.2 | 2.4 | 9.0 |
| Buffer | <0.000 | <0.000 | N/A | N/A |
| #13 | 0.1 | 0.1 | 0.1 | 2.6 |
| #7 | 1.6 | 1.6 | 1.6 | 0.0 |
| #9 | 1.3 | 2.1 | 1.7 | 33.3 |
| #5 | 2.5 | 2.3 | 2.4 | 5.9 |
| #3 | 2.8 | 3.5 | 3.2 | 15.7 |
| #14 | 3.4 | 2.9 | 3.2 | 11.2 |
| #6 | 3.4 | 3.5 | 3.5 | 2.0 |
| #8 | 4.8 | 5.1 | 5.0 | 4.3 |
| #10 | 12.8 | 11.9 | 12.4 | 5.2 |
| #181 | 12.9 | 12.8 | 12.9 | 0.6 |
| #1 | 14.6 | 14.1 | 14.4 | 2.5 |
| #12 | 60.8 | 57.4 | 59.1 | 4.1 |
| #11 | 68.2 | 77.4 | 72.8 | 8.9 |
| #4 | 94.6 | 89.0 | 91.8 | 4.3 |
| #2 | 116.1 | 88.2 | 102.2 | 19.3 |
| #20 | 155.6 | 89.5 | 122.6 | 38.1 |

### Example 2—Examples of Certain Embodiments

A1. A method for quantitative detection of an antibody of interest comprising:

(a) obtaining a surface coated with a binding agent, wherein the binding agent optionally is streptavidin;

(b) adding a first antibody, wherein the first antibody is conjugated with a ligand that binds to the binding agent, wherein the ligand is optionally biotin, and wherein a first antibody-ligand-binding agent complex forms;

(c) adding a secondary antibody such that a secondary antibody-first antibody-ligand-binding agent complex forms;

(d) measuring the bound secondary antibody;

(e) repeating steps (a)-(d) at various concentrations of first antibody to generate a standard curve; and

(f) using the standard curve of step e) to quantify the presence of the antibody of interest in a sample wherein the quantification of the antibody of interest in the sample comprises the steps of:
(i) adding an antigen for the antibody of interest to the surface coated with the binding agent, wherein the antigen is conjugated to the ligand, and wherein an antigen-ligand-binding agent complex forms;
(ii) adding a sample comprising the antibody of interest to the surface such that an antibody of interest-antigen-ligand-binding agent complex forms;
(iii) adding the secondary antibody to the surface such that a secondary antibody-antibody of interest-antigen-ligand-binding agent complex forms;
(iv) measuring bound secondary antibody; and
(v) quantifying the amount of bound antibody of interest using the standard curve of step e).
   A2. The method of A1, wherein the first antibody is purified IgG.
   A3. The method of A1 and A2, wherein the secondary antibody is labeled with a detectable moiety.
   A4. The method of any of the preceding or subsequent embodiments, wherein the detectable moiety is an enzyme.
   A5. The method of any of the preceding or subsequent embodiments, wherein the detectable moiety is a fluorophore.
   A6. The method of any of the preceding or subsequent embodiments, wherein the antibody of interest is an anti-receptor binding domain (RBD) IgG.
   A7. The method of any of the preceding or subsequent embodiments, wherein the antibody of interest is anti-SARS-CoV-2 RBD IgG.
   A8. The method of any of the preceding or subsequent embodiments, wherein the sample is a serum sample.
   A9. The method of any of the preceding or subsequent embodiments, wherein the sample is a dried blood spot.
   A10. The method of any of the preceding or subsequent embodiments, wherein the subject is suspected to have been exposed to SARS-CoV-2.
   A11. The method of embodiment A10, wherein the sample is taken within three weeks of the subject's suspected exposure to SARS-CoV-2.
   A12. The method of embodiment A10, wherein the sample is taken more than two weeks after the subject's suspected exposure to SARS-CoV-2.
   A13. The method of any of the preceding or subsequent embodiments, wherein the subject is a human subject.
   A14. The method of any of the preceding or subsequent embodiments, wherein the antigen for the antibody of interest is a RBD.
   A15. The method of embodiment A14, wherein the receptor binding domain is the RBD of SARS-CoV-2.
   A16. The method of embodiment A4, wherein the detectable moiety is horseradish peroxidase (HRP).
   A17. The method of any of the preceding or subsequent embodiments, wherein the method does not detect the presence of antibodies reactive to a human coronavirus selected from the group consisting of: 229E, NL63, OC43, HKU1, and MERS-CoV.
   A18. The method of any of the preceding or subsequent embodiments, further comprising determining that a person has mounted an immune response to SARS-CoV-2.
   A19. The method of any of the preceding embodiments, wherein the standard curve used for quantifying the antibody of interest is developed using an immunoglobulin that does not specifically recognize the antigen(s) recognized by the antibody of interest but is of the same immunoglobulin class as the antibody of interest.
   A20. The method of any of the preceding or subsequent embodiments, further comprising determining if a sample from a convalescent subject contains enough antibody of interest for the subject's plasma to be used as donor plasma.
   A21. The method of any of the preceding embodiments, further comprising using the quantified amount of antibody of interest to determine a protective immunity level of the antibody of interest.
   B1. A method for quantitative detection of an antibody of interest in a sample comprising:

(a) adding an antigen recognized by the antibody of interest to a surface comprising a binding agent, wherein the binding agent binds a ligand conjugated to the antigen;

(b) adding the sample to the surface such that the antibody of interest binds to the antigen to form an antibody of interest-antigen-ligand-binding agent complex;

(c) adding a secondary antibody to the surface that binds the antibody of interest to form a secondary antibody-antibody of interest-antigen-ligand-binding agent complex;

(d) detecting the complexed secondary antibody; and

(e) quantifying the amount of bound antibody of interest based on the detected secondary antibody, wherein quantifying comprises using a standard curve generated by detecting complexes of secondary antibody-a first antibody at varying concentrations of ligand-binding agent complexes formed under similar assay conditions.

B2. The method of B1, wherein the first antibody is purified IgG.
B3. The method of B1 and B2, wherein the secondary antibody is labeled with a detectable moiety.
B4. The method of any of the preceding embodiments, wherein the detectable moiety is an enzyme.
B5. The method of any of the preceding embodiments, wherein the detectable moiety is a fluorophore.
B6. The method of any of the preceding or subsequent embodiments, wherein the antibody of interest is an anti-receptor binding domain (RBD) IgG.
B7. The method of any of the preceding or subsequent embodiments, wherein the antibody of interest is anti-SARS-CoV-2 RBD IgG.
B8. The method of any of the preceding or subsequent embodiments, wherein the sample is a serum sample.
B9. The method of any of the preceding or subsequent embodiments, wherein the sample is a dried blood spot.
B10. The method of any of the preceding or subsequent embodiments, wherein the subject is suspected to have been exposed to SARS-CoV-2.
B11. The method of embodiment B10, wherein the sample is taken within three weeks of the subject's suspected exposure to SARS-CoV-2.
B12. The method of embodiment B10, wherein the sample is taken more than two weeks after the subject's suspected exposure to SARS-CoV-2.
B13. The method of any of the preceding or subsequent embodiments, wherein the subject is a human subject.
B14. The method of any of the preceding or subsequent embodiments, wherein the antigen for the antibody of interest is a RBD.
B15. The method of embodiment B14, wherein the receptor binding domain is the RBD of SARS-CoV-2.
B16. The method of embodiment B4, wherein the detectable moiety is horseradish peroxidase (HRP).
B17. The method of any of the preceding or subsequent embodiments, wherein the method does not detect the presence of antibodies reactive to a human coronavirus selected from the group consisting of: 229E, NL63, OC43, HKU1, and MERS-CoV.
B18. The method of any of the preceding embodiments, further comprising determining that a person has mounted an immune response to SARS-CoV-2.
B19. The method of any of the preceding embodiments, wherein the standard curve used for quantifying the antibody of interest is developed using an immunoglobulin that does not specifically recognize the antigen(s) recognized by the antibody of interest but is of the same immunoglobulin class as the antibody of interest.
B20. The method of any of the preceding or subsequent embodiments, further comprising determining if a sample from a convalescent subject contains enough antibody of interest for the subject's plasma to be used as donor plasma.
B21. The method of any of the preceding embodiments, further comprising using the quantified amount of antibody of interest to determine a protective immunity level of the antibody of interest.
C1. A method for quantitative detection of an antibody of interest in a sample comprising:

(a) obtaining a surface coated with a binding agent, wherein the binding agent is optionally streptavidin;

(b) adding to the surface an antigen that is recognized by the antibody of interest, wherein the antigen is conjugated to a ligand that binds to the binding agent, wherein the ligand is optionally biotin;

(c) adding the sample to the surface such that the antibody of interest present in the sample will bind to the antigen;

(d) detecting the bound antibody of interest; and

(e) quantifying the amount of bound antibody of interest.

C2. The method of embodiment C1, wherein the bound antibody of interest is detected using a secondary antibody.
C3. The method of embodiment C2, wherein the secondary antibody is labeled with a detectable moiety.
C4. The method of embodiment C3, wherein the detectable moiety is an enzyme.
C5. The method of embodiment C3, wherein the detectable moiety is a fluorophore.
C6. The method of any of the preceding or subsequent embodiments, wherein the quantification step utilizes a standard curve.
C7. The method of any of the preceding or subsequent embodiments, wherein the antibody of interest is an anti-receptor binding domain (RBD) IgG.
C8. The method of any of the preceding or subsequent embodiments, wherein the antibody of interest is anti-SARS-CoV-2 RBD IgG.
C9. The method of any of the preceding or subsequent embodiments, wherein the sample is a serum sample.
C10. The method of any of the preceding or subsequent embodiments, wherein the sample is a dried blood spot.
C11. The method of any of the preceding or subsequent embodiments, wherein the subject is suspected to have been exposed to SARS-CoV-2.
C12. The method of embodiment C11, wherein the sample is taken within three weeks of the subject's suspected exposure to SARS-CoV-2.
C13. The method of embodiment C11, wherein the sample is taken more than two weeks after the subject's suspected exposure to SARS-CoV-2.
C14. The method of any of the preceding or subsequent embodiments, wherein the subject is a human subject.
C15. The method of any of the preceding or subsequent embodiments, wherein the antigen for the antibody of interest is a RBD.
C16. The method of embodiment C15, wherein the receptor binding domain is the RBD of SARS-CoV-2.
C17. The method of embodiment C4, wherein the detectable moiety is horseradish peroxidase (HRP).
C18. The method of any of the preceding or subsequent embodiments, wherein the method does not detect the presence of antibodies reactive to a human coronavirus selected from the group consisting of: 229E, NL63, OC43, HKU1, and MERS-CoV.
C19. The method of any of the preceding embodiments, further comprising determining that a person has mounted an immune response to SARS-CoV-2.
C20. The method of any of the preceding embodiments, wherein a standard curve used for quantifying the antibody of interest is developed using an immunoglobulin that does not specifically recognize the antigen(s) recognized by the antibody of interest but is of the same immunoglobulin class as the antibody of interest.
C21. The method of any of the preceding or subsequent embodiments, further comprising determining if a sample from a convalescent subject contains enough antibody of interest for the subject's plasma to be used as donor plasma.
C22. The method of any of the preceding embodiments, further comprising using the quantified amount of antibody of interest to determine a protective immunity level of the antibody of interest.
D1. A method for developing an assay for quantitative detection of an antibody of interest comprising:

(a) obtaining a surface coated with a binding agent, wherein the binding agent optionally is streptavidin;

(b) adding a first antibody, wherein the first antibody is conjugated with a ligand that binds to the binding agent, wherein the ligand is optionally biotin, and wherein a first antibody-ligand-binding agent complex forms;

(c) adding a secondary antibody such that a secondary antibody-first antibody-ligand-binding agent complex forms;

(d) measuring the bound secondary antibody;

(e) repeating steps (a)-(d) at various concentrations of first antibody to generate a standard curve; and

(f) using the standard curve of step e) to quantify the presence of the antibody of interest in a sample wherein the quantification of the antibody of interest in the sample comprises the steps of:
(i) adding an antigen for the antibody of interest to the surface coated with the binding agent, wherein the antigen is conjugated to the ligand, and wherein an antigen-ligand-binding agent complex forms;
(ii) adding a sample comprising the antibody of interest to the surface such that an antibody of interest-antigen-ligand-binding agent complex forms;
(iii) adding the secondary antibody to the surface such that a secondary antibody-antibody of interest-antigen-ligand-binding agent complex forms;
(iv) measuring bound secondary antibody; and
(v) quantifying the amount of bound antibody of interest using the standard curve of step e).
   D2. The method of any of the preceding embodiments, wherein the standard curve used for quantifying the antibody of interest is developed using an immunoglobulin that does not specifically recognize the antigen(s) recognized by the antibody of interest but is of the same immunoglobulin class as the antibody of interest.
   E1. A system for quantitative detection of an antibody of interest comprising stations and/or components for performing any of the steps of the methods of any of the preceding embodiments.
   E2. The system of any of the preceding or subsequent embodiments, wherein a standard curve used for quantifying the antibody of interest is developed using an immunoglobulin that does not specifically recognize the antigen(s) recognized by the antibody of interest but is of the same immunoglobulin class as the antibody of interest.
   E3. The system of any of the preceding or subsequent embodiments comprising a surface coated with a binding agent.
   E4. The system of any of the preceding or subsequent embodiments comprising a first antibody complexed to a ligand for the binding agent.
   E5. The system of any of the preceding or subsequent embodiments comprising a secondary antibody.
   E6. The system of any of the preceding or subsequent embodiments comprising a station and/or components for generating a standard curve by any of the preceding method embodiments.
   E7. The system of any of the preceding or subsequent embodiments comprising reagents for conjugating an antibody of interest to the ligand.
   E8. The system of any of the preceding or subsequent embodiments comprising a station and/or components for quantitating the antibody of interest using the standard curve by any of the preceding method embodiments.
   F1. A kit for quantitative detection of an antibody of interest comprising at least one component for quantitative detection of an antibody of interest by any of the steps of any of the methods of any of the preceding embodiments.
   F2. The kit of any of the preceding or subsequent embodiments, wherein a standard curve used for quantifying the antibody of interest is developed using an immunoglobulin that does not specifically recognize the antigen(s) recognized by the antibody of interest but is of the same immunoglobulin class as the antibody of interest.
   F3. The kit of any of the preceding or subsequent embodiments comprising a surface coated with a binding agent.
   F4. The kit of any of the preceding or subsequent embodiments comprising a first antibody complexed to a ligand for the binding agent.
   F5. The kit of any of the preceding or subsequent embodiments comprising a secondary antibody.
   F6. The kit of any of the preceding or subsequent embodiments comprising instructions for generating a standard curve by methods described herein.
   F7. The kit of any of the preceding or subsequent embodiments comprising reagents for conjugating an antibody of interest to the ligand.
   F8 The kit of any of the preceding or subsequent embodiments comprising instructions for quantitating the antibody of interest using the standard curve.

That which is claimed is:

1. A method for quantitative detection of an antibody of interest comprising:
(a) obtaining a surface coated with a binding agent, wherein the binding agent optionally is streptavidin;
(b) adding a first antibody, wherein the first antibody is conjugated with a ligand that binds to the binding agent, wherein the ligand is optionally biotin, and wherein a first antibody-ligand-binding agent complex forms;
(c) adding a secondary antibody such that a secondary antibody-first antibody-ligand-binding agent complex forms;
(d) measuring the bound secondary antibody;
(e) repeating steps a)-d) at various concentrations of first antibody to generate a standard curve; and
(f) using the standard curve of step e) to quantify the presence of the antibody of interest in a sample wherein the quantification of the antibody of interest in the sample comprises the steps of:
(i) adding an antigen for the antibody of interest to the surface coated with the binding agent, wherein the antigen is conjugated to the ligand, and wherein an antigen-ligand-binding agent complex forms;
(ii) adding a sample comprising the antibody of interest to the surface such that an antibody of interest-antigen-ligand-binding agent complex forms;
(iii) adding the secondary antibody to the surface such that a secondary antibody-antibody of interest-antigen-ligand-binding agent complex forms;
(iv) measuring bound secondary antibody; and
(v) quantifying the amount of bound antibody of interest using the standard curve of step e).

2. The method of claim 1, wherein the first antibody is purified IgG.

3. The method of claim 1, wherein the secondary antibody is labeled with a detectable moiety.

4. The method of claim 1, wherein the antibody of interest is anti-SARS-CoV-2 RBD IgG.

5. The method of claim 1, wherein the sample is a serum sample.

6. The method of claim 1, wherein the subject is suspected to have been exposed to SARS-CoV-2.

7. The method of claim 1, wherein the antigen for the antibody of interest is a RBD.

8. The method of claim 7, wherein the receptor binding domain is the RBD of SARS-CoV-2.

9. The method of claim 1, wherein the method does not detect the presence of antibodies reactive to a human coronavirus selected from the group consisting of: 229E, NL63, OC43, HKU1, and MERS-CoV.

10. A method for quantitative detection of an antibody of interest in a sample comprising:
(a) adding an antigen recognized by the antibody of interest to a surface comprising a binding agent, wherein the binding agent binds a ligand conjugated to the antigen;
(b) adding the sample to the surface such that the antibody of interest binds to the antigen to form an antibody of interest-antigen-ligand-binding agent complex;
(c) adding a secondary antibody to the surface that binds the antibody of interest to form a secondary antibody-antibody of interest-antigen-ligand-binding agent complex;
(d) detecting the complexed secondary antibody; and
(e) quantifying the amount of bound antibody of interest based on the detected secondary antibody, wherein quantifying comprises using a non-antigen specific standard curve generated by detecting complexes of secondary antibody-a first antibody at varying concentrations of ligand-binding agent complexes formed under similar assay conditions, wherein the first antibody is of the same class of antibody as the antibody of interest.

11. The method of claim 10, wherein the first antibody is purified IgG.

12. The method of claim 10, wherein the secondary antibody is labeled with a detectable moiety.

13. The method of claim 12, wherein the detectable moiety is an enzyme.

14. The method of claim 10, wherein the antibody of interest is anti-SARS-CoV-2 RBD IgG.

15. The method of claim 10, wherein the sample is a serum sample.

16. The method of claim 10, wherein the subject is suspected to have been exposed to SARS-CoV-2.

17. The method of claim 10, wherein the antigen for the antibody of interest is a RBD.

18. The method of claim 10, wherein the method does not detect the presence of antibodies reactive to a human coronavirus selected from the group consisting of: 229E, NL63, OC43, HKU1, and MERS-CoV.

19. A system for quantitative detection of an antibody of interest comprising stations for:
(a) obtaining a surface coated with a binding agent, wherein the binding agent optionally is streptavidin;
(b) adding a first antibody, wherein the first antibody is conjugated with a ligand that binds to the binding agent, wherein the ligand is optionally biotin, and wherein a first antibody-ligand-binding agent complex forms;
(c) adding a secondary antibody such that a secondary antibody-first antibody-ligand-binding agent complex forms;
(d) measuring the bound secondary antibody;
(e) repeating steps (a)-(d) at various concentrations of first antibody to generate a standard curve; and
(f) using the standard curve of step e) to quantify the presence of the antibody of interest in a sample wherein the quantification of the antibody of interest in the sample comprises the steps of:
(i) adding an antigen for the antibody of interest to the surface coated with the binding agent, wherein the antigen is conjugated to the ligand, and wherein an antigen-ligand-binding agent complex forms;
(ii) adding a sample comprising the antibody of interest to the surface such that an antibody of interest-antigen-ligand-binding agent complex forms;
(iii) adding the secondary antibody to the surface such that a secondary antibody-antibody of interest-antigen-ligand-binding agent complex forms;
(iv) measuring bound secondary antibody; and
(v) quantifying the amount of bound antibody of interest using the standard curve of step e).

20. The method of claim 19, wherein the antibody of interest is anti-SARS-CoV-2 RBD IgG.
